(19) United States
(12) Patent Application Publication
(10) Pub. No.: US 2015/0275285 A1
(43) Pub. Date: Oct. 1, 2015

(54) COMPOSITIONS AND METHODS OF NUCLEIC ACID PREPARATION AND ANALYSES

(71) Applicant: Yilin ZHANG, (US)

(72) Inventor: Yilin Zhang, Hayward, CA (US)

(21) Appl. No.: 14/646,900

(22) PCT Filed: Dec. 2, 2013
(86) PCT No.: PCT/US2013/072705

§ 371 Date: May 22, 2015

Related U.S. Application Data
(60) Provisional application No. 61/732,823, filed on Dec. 3, 2012.

(51) Int. Cl. C12Q 1/68

(52) U.S. Cl. CPC C12Q 1/6855; C12Q 1/6874

(57) ABSTRACT

The present invention provides methods of generating single-stranded polynucleotides comprising use of adaptor sequence(s), single-stranded polynucleotide amplification and a primer comprising BJSA. Methods of analysing one or more regions on a desired polynucleotide using probes and single-stranded polynucleotides are also provided. Also provided are kits and compositions useful for these methods.

### CROSS-REFERENCE TO RELATED APPLICATIONS

This application claims priority benefit from U.S. Provisional Patent Application No. 61/732,823 filed on Dec. 3, 2012 which is incorporated by reference in its entirety.

### TECHNICAL FIELD

This application relates generally to the fields of nucleic acid sample preparation and sequencing.

### BACKGROUND

Nucleic acid sequence analysis tools are fundamental for the identification of gene alterations, which in turn are useful for diagnosing genetic diseases, predicting responsiveness to drug treatments, and analyzing pharmacogenomics of drugs. Because sequencing analyses frequently involve the determination of rare genetic alterations in a limited amount of sample, sensitivity has been a big challenge. This is particularly true when analyzing somatic mutations in a tissue sample (such as a cancer sample), which frequently contains normal cells mixed with cells harboring the mutation.

To increase sensitivity, various nucleic acid amplification methods are used. The most commonly used amplification method is polymerase chain reaction (“PCR”), which involves multiple cycles of amplifications using the Taq polymerase. Because of the inherent fidelity issues with Taq polymerases, the PCR methods frequently generate artificial mutations, which may mask the real mutations to be analyzed and make it extremely difficult to detect rare mutations in the sample. As a consequence, the accuracy of the nucleic acid methods may be compromised.

The human genomic DNA is complex and has many repetitive sequences. This presents additional challenges for sequence analyses. First, polynucleotides of interest may be significantly under-represented among the mixture of polynucleotides. Second, the cost of analyzing the complex DNA sample can be prohibitively expensive, particularly in the context of analyzing genomic DNA and detecting multiple genetic mutations. While many next generation sequencing methods have been developed, there remains a need for sensitive, accurate, and efficient methods for nucleic acid preparation and sequencing analyses.

All references cited herein, including patent applications and publications, are incorporated by reference in their entirety.

### SUMMARY OF THE INVENTION

The present application in one aspect provides a method of generating single-stranded polynucleotides comprising asymmetric adaptor sequences, comprising: i) ligating one or more DNA fragments to: a) a first adaptor comprising a tag; and b) a second adaptor comprising a recognition sequence; ii) selecting DNA fragments comprising the first adaptor based on the presence of the tag; iii) amplifying the DNA fragments selected from step ii) by single-strand polynucleotide amplification using a primer comprising RNA and hybridizing to the recognition sequence, thereby selectively amplifying DNA fragments comprising the second adaptor. In some embodiments, the one or more DNA fragments are generated by fragmenting a double-stranded target DNA (such as genomic DNA). In some embodiments, one strand of the DNA fragment selected from step ii) is physically separated from its complementary strand before it is used as a template for the single-strand polynucleotide amplification.

In some embodiments, there is provided a method of generating single-stranded polynucleotides comprising an adaptor sequence from a double-stranded target DNA, comprising: i) cleaving the double-stranded target DNA with a restriction endonuclease to generate DNA fragments having a 5′ or 3′ overhang; ii) ligating the DNA fragments with an adaptor that comprises a) a single-stranded 5′or 3′ overhang complementary to the 5′ or 3′ overhang of the DNA fragments and b) a recognition sequence; and iii) amplifying the DNA fragments ligated to the adaptor by single-strand polynucleotide amplification using a primer comprising RNA and hybridizing to the recognition sequence. In some embodiments, the DNA fragments ligated to the adaptor are further fragmented before they are subjected to single-strand polynucleotide amplification.

In some embodiments according to any one of the embodiments described in the paragraphs above, the method further comprises preparing a library of polynucleotides from said single-stranded polynucleotides.

In some embodiments according to any one of the embodiments described in the paragraphs above, the method further comprises immobilizing the single-stranded polynucleotides on a solid support.

In some embodiments according to any one of the embodiments described in the paragraph above, the method further comprises analyzing (such as sequencing) said single-stranded polynucleotides.

In some embodiments, there is provided a method of analyzing (such as sequencing) one or more desired regions on a target polynucleotide, wherein the one or more desired regions are hybridizable to a set of probes, comprising: 1) contacting a population of single-stranded polynucleotides generated from said target polynucleotide with the set of probes; 2) separating polynucleotides that are bound to the probes from the rest of the polynucleotides, wherein polynucleotides comprising the one or more desired regions are enriched; and 3) analyzing (such as sequencing) the separated polynucleotides. In some embodiments, the population of single-stranded polynucleotides is generated from said target polynucleotide by single-strand polynucleotide amplification using a primer comprising RNA and DNA fragments generated from said target polynucleotide as template. In some embodiments, the one or more desired regions are regions where oncogenes are located. In some embodiments, the set of probes comprises at least about 10 different polynucleotide probes. In some embodiments, the set of polynucleotide probes comprises at least about 50 different polynucleotide probes. In some embodiments, the target polynucleotide is RNA. In some embodiments, the target polynucleotide is a double-stranded DNA (such as genomic DNA). In some embodiments, the population of single-stranded polynucleotides is generated by the methods described in the paragraphs above.

In some embodiments according to any one of the embodiments described above, the single-strand polynucleotide amplification comprise: a) extending the primer comprising RNA in a complex comprising: i) the DNA fragment to be amplified and ii) the primer comprising RNA, wherein the primer comprising RNA is hybridized to the DNA fragment to be amplified; and b) cleaving the RNA portion of the primer with an enzyme that cleaves RNA from an RNA/DNA hybrid such that another primer comprising RNA hybridizes to the DNA fragment and repeats primer extension by strand displacement; whereby multiple copies of single-stranded polynucleotides are generated.

In some embodiments according to any one of the embodiments described above, the single-strand polynucleotide amplification comprises use of an RNA primer. In some embodiments, the single-strand polynucleotide amplification comprises use of a DNA-RNA composite primer. In some embodiments, the extension is carried out by a DNA polymerase selected from the group consisting of a strand displacing DNA polymerase, a high-fidelity DNA polymerase, a polymerase that has proofreading activity, a T7 DNA polymerase, and an E. coli DNA polymerase. In some embodiments, the enzyme that cleaves RNA from the RNA/DNA hybrid is RNase H or RNase I.

In some embodiments, there is provided a kit comprising i) a first adaptor comprising a tag; and b) a second adaptor comprising a recognition sequences, and iii) a primer that hybridizes to the recognition sequence. In some embodiments, the kit further comprises a ligand that binds to the tag. In some embodiments, the kit further comprises a solid support. In some embodiments, the primer comprises RNA. In some embodiments, the primer is an RNA primer. In some embodiments, the primer is a DNA/RNA composite primer. In some embodiments, the primer is about 5 to about 30 nucleotides. In some embodiments, the kit further comprises an enzyme that cleaves RNA from an RNA/DNA hybrid (such as RNase H or RNase I). In some embodiments, the kit further comprises a DNA polymerase, such as a DNA polymerase selected from the group consisting of a strand displacing DNA polymerase, a high-fidelity DNA polymerase, a polymerase that has proofreading activity, a T7 DNA polymerase, and an E. coli DNA polymerase. In some embodiments, the kit further comprises a DNA ligase. In some embodiments, the kit further comprises one or more probes. In some embodiments, the kit further comprises an instruction for carrying out any one of the methods described herein.

### DESCRIPTION OF THE DRAWINGS

FIG. 1 depicts one exemplary method of processing DNA using asymmetric adaptors.

FIG. 2 depicts one exemplary method of processing DNA using restriction enzyme digestion.

### DETAILED DESCRIPTION

The present application provides methods of nucleic acid preparation and analysis which allow sensitive, accurate, and efficient determination of nucleic acid sequences. The methods generally involve the generation of single-stranded polynucleotides by amplifying a target polynucleotide using single-strand polynucleotide amplification. The target nucleic acids can be processed, for example by adding one or more adaptors, and nucleic acids comprising the one or more adaptors can be selected and used for the generation of the single-stranded polynucleotides. The single-stranded polynucleotides can be further enriched for polynucleotides containing regions of interest by using a set of probes that hybridize with regions of interest on the single-stranded polynucleotides.

Thus, the present application in one aspect provides methods of generating single-stranded polynucleotides comprising one or more adaptors.

In another aspect, there are provided methods of analyzing one or more desired regions on a target polynucleotide.

In another aspect, there are provided kits, compositions, and articles of manufacture useful for methods described herein.

### I. Definitions

“Single-strand polynucleotide amplification” used herein refers to the synthesis of multiple copies of single-stranded daughter strands by repeatedly extending a single primer over single-stranded template nucleic acid that comprises a target polynucleotide sequence. The newly synthesized nucleic acid molecules cannot serve as templates for the production of additional nucleic acid molecules during subsequent primer extension reactions.

“Amplification,” as used herein, generally refers to the process of producing two or more copies of a desired sequence. “Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs.

“Oligonucleotide,” as used herein, generally refers to short, generally single-stranded, generally synthetic polynucleotides that are generally, but not necessarily, no more than about 200 nucleotides in length. The terms “oligonucleotide” and “polynucleotide” are not mutually exclusive. The description above for polynucleotides is equally and fully applicable to oligonucleotides.

“Fragmenting” a polynucleotide used herein refers to breaking the polynucleotides into different polynucleotide fragments. Fragmenting can be achieved, for example, by shearing or by enzymatic reactions.

A “primer” is generally a short single-stranded polynucleotide, generally with a free 3′-OH group, that binds to a target of interest by hybridizing with a target sequence, and thereafter promotes polymerization of a polynucleotide complementary to the target.

The term “tag” as used herein refers to a moiety that can be used to separate a molecule to which the tag is attached to from other molecules that do not contain the tag.

The term “terminal nucleotide,” as used herein refers to the nucleotide at either the 5′ or 3′ end of a nucleic acid molecule.

“Hybridization” and “annealing” refer to a reaction in which one or more polynucleotides react to form a complex that is stabilized via hydrogen bonding between the bases of the nucleotide residues. The hydrogen bonding may occur by Watson-Crick base pairing, Hoogstein binding, or by any other sequence specific manner.

An “adaptor” used herein refers to an oligonucleotide that can be joined to a polynucleotide fragment.

The term “ligation” as used herein, with respect to two polynucleotides, such as an adaptor and a polynucleotide fragment, refers to the covalent attachment of two separate polynucleotides to produce a single larger polynucleotide with a contiguous backbone.

The term “3′” generally refers to a region or position in a polynucleotide or oligonucleotide that is downstream of another region or position in the same polynucleotide or oligonucleotide.

The term “5′” generally refers to a region or position in a polynucleotide or oligonucleotide that is upstream from another region or position in the same polynucleotide or oligonucleotide.

A “5′ overhang” is a stretch of unpaired nucleotides that extend past the 5′ end of a double-stranded nucleic acid molecule. For example, a 5′ overhang can be a single unpaired nucleotide, or it can be at least 5, 10, 15 or more than 15 nucleotides long. For example, a primer can comprise, e.g., 5-25 nucleotides that are not complementary to, e.g., sequences present in a template strand and/or target polynucleotide sequence. In other words, the nucleotides of the 5′ overhang do not hybridize to the target polynucleotide sequence under conditions in which other portion(s) of the primer hybridizes to the target polynucleotide.

A “3′ overhang” is a stretch of unpaired nucleotides that extend past the 3′ end of a double-stranded nucleic acid molecule. For example, a 3′ overhang can be a single unpaired nucleotide, or it can be at least 5, 10, 15 or more than 15 nucleotides long. For example, a primer can comprise, e.g., 5-25 nucleotides that are not complementary to, e.g., sequences present in a template strand and/or target polynucleotide sequence. In other words, the nucleotides of the 3′ overhang do not hybridize to the target polynucleotide sequence under conditions in which other portion(s) of the primer hybridizes to the target polynucleotide.

The term “target polynucleotide” as used herein refers to a polynucleotide that contains one or more sequences that are of interest and under study.

An “array” used herein includes arrangement of spatially or optically addressable regions bearing nucleic acids or other molecules. When the arrays are arrays of nucleic acids, the nucleic acids may be physically adsorbed, chemically adsorbed, or covalently attached to the arrays at any point or points along the nucleic acid chain.

The term “determining,” “measuring,” “evaluating,” “assessing,” “assaying,” and “analyzing” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

As used herein, the term “single nucleotide polymorphism,” or “SNP” for short, refers to the alteration of a single nucleotide at a specific position in a genomic sequence, resulting in two or more alternative alleles that occur in a population at appreciable frequency (e.g., at least 1% in a population).

The term “denaturing” as used herein refers to the separation of a nucleic acid duplex into two single-strands.

The term “enrichment” refers to the process of increasing the relative abundance of particular nucleic acid sequences in a sample relative to the level of nucleic acid sequences as a whole initially present in said sample before treatment. Thus the enrichment step provides a relative percentage or fractional increase, rather than directly increasing, for example, the absolute copy number of the nucleic acid sequences of interest. After the step of enrichment, the sample to be analyzed may be referred to as an enriched, or selected polynucleotide.

As used herein, the “complexity” of a nucleic acid sample refers to the number of different unique sequences present in that sample. A sample is considered to have “reduced complexity” if it is less complex than the nucleic acid sample from which it is derived.

As used herein, “solid support” refers to a solid or semisolid material which has the property, either inherently or through attachment of some component conferring the property (e.g., an antibody, streptavidin, nucleic acid, or other binding ligands), of binding to a tag. Such binding may be direct or indirect. Examples of solid support include, but are not limited to, nitrocellulose and nylon membranes, agarose or cellulose based beads (e.g., Sepharose) and paramagnetic beads.

As used herein, the term “library” refers to a collection of nucleic acid sequences.

As used herein, the term “hybridize specifically” means that nucleic acids hybridize with a nucleic acid of complementary sequence. As used herein, a portion of a nucleic acid molecule may hybridize specifically with a complementary sequence on another nucleic acid molecule. That is, the entire length of a nucleic acid sequence does not necessarily need to hybridize for a portion of such sequence to be “specifically hybridized” to another molecule, there may be, for example, a stretch of nucleotides at the 5′ end of a molecule that do not hybridize while a stretch at the 3′ end of the same molecule is specifically hybridized to another molecule.

A “portion” or “region,” used interchangeably herein, of a polynucleotide or oligonucleotide is a contiguous sequence of 2 or more bases. In other embodiments, a region or portion is at least about any of 3, 5, 10, 15, 20, 25 contiguous nucleotides.

Sequence “mutation,” as used herein, refers to any sequence alteration in a sequence of interest in comparison to a reference sequence. A reference sequence can be a wild type sequence or a sequence to which one wishes to compare a sequence of interest. A sequence mutation includes single nucleotide changes, or alterations of more than one nucleotide in a sequence, due to mechanisms such as substitution, deletion or insertion. Single nucleotide polymorphism (SNP) is an example of a sequence mutation as used herein.

A “complex” is a group of molecules comprising of any two or more of, e.g., a polypeptide, a nucleic acid, a primer, etc., that assemble to function together to carry out a specific reaction, e.g. a primer extension reaction. For example, in the present invention, a complex can comprise, e.g., a DNA template strand and an RNA primer that is hybridized to the DNA strand. The complex can optionally comprise a DNA polymerase that extends the RNA primer. A complex may or may not be stable and may be directly or indirectly detected. For example, as is described herein, given certain components of a reaction, and the type of product(s) of the reaction, existence of a complex can be inferred. For purposes of this invention, a complex is generally an intermediate with respect to formation the final amplification product(s), i.e., daughter strands.

As used herein, “cleaving” or “to cleave” refers to enzymatic digestion, e.g., of the RNA portion of an RNA: DNA hybrid.

A nucleic acid or primer is “complementary” to another nucleic acid when at least two contiguous bases of, e.g., a first nucleic acid or a primer, can combine in an antiparallel association or hybridize with at least a subsequence of a second nucleic acid to form a duplex. In some embodiments, complementarity between e.g., a primer and a target polynucleotide sequence, is not 100% perfect.

A “primer extension reaction” refers to a molecular reaction in which a nucleic acid polymerase adds one or more nucleotides to the 3′ terminus of a primer that is hybridized to a target polynucleotide sequence in a template-specific manner, i.e., wherein the daughter strand produced by the primer extension reaction is complementary to the target polynucleotide sequence. Extension does not only refer to the first nucleotide added to the 3′ terminus of a primer, but also includes any further extension of a polynucleotide formed by the extended primer.

A “random primer” as used herein, is a primer that comprises a sequence that is based on a statistical expectation (or an empirical observation) that the sequence of the random primer is hybridizable (under a given set of conditions) to one or more sequences a nucleic acid sample, e.g., a genomic DNA, a population of RNAs, etc. The sequence of a random primer may or may not be naturally-occurring, or may or may not be present in a pool of sequences in a sample of interest. The amplification of a plurality of different daughter strands in a single reaction mixture would generally, but not necessarily, employ a multiplicity, preferably a large multiplicity, of random primers. As is well understood in the art, a “random primer” can also refer to a primer that is a member of a population of primers (a plurality of random primers) which collectively are designed to hybridize to a desired and/or a significant number of target sequences. A random primer may hybridize at a plurality of sites on a template nucleic acid. The use of random primers provides a method for generating primer extension products complementary to a target polynucleotide which does not require prior knowledge of the exact sequence of the target.

A “reaction mixture” is an assemblage of components (e.g., one or more polypeptides, nucleic acids, and/or primers), which, under suitable conditions, react to carry out a specific reaction, e.g. a primer extension reaction.

A “termination polynucleotide sequence” or a “termination sequence”, as used interchangeably herein, is a polynucleotide sequence which promotes the termination of a primer extension reaction by diverting or blocking further extension of the daughter strand beyond a specified position on the target polynucleotide sequence. A termination sequence comprises a portion (or region) that generally hybridizes to the target polynucleotide sequence at a location 3′ to the primer hybridization site. The portion of termination sequence capable of hybridizing to the target polynucleotide sequence may or may not encompass the entire termination sequence. For example, a termination sequence can be, e.g., an oligonucleotide that binds, generally with high affinity, to the template nucleic acid at a location 5′ to the termination site and 3′ to the primer hybridization site. Its 3′ end may or may not be blocked for extension by DNA polymerase. The site, point or region of the target polynucleotide that is last replicated by the DNA polymerase before the termination of a primer extension reaction is a “termination site” or “termination point”.

It is understood that aspect and embodiments of the invention described herein include “consisting” and/or “consisting essentially of aspects and embodiments.

As used herein, the singular form “a”, “an”, and “the” includes plural references unless indicated otherwise.

As is understood by one skilled in the art, reference to “about” a value or parameter herein includes (and describes) embodiments that are directed to that value or parameter per se. For example, description referring to “about X” includes description of “X”.

### I. Methods of Generating Single-Stranded Polynucleotides Comprising Adaptor Sequences

The present application in one aspect provides methods of generating single-stranded polynucleotides comprising adaptor sequences.

In some embodiments, the method uses asymmetric adaptors, i.e., adaptors having different sequences. The adaptors are ligated to DNA fragments such that at least some of the DNA fragments comprise a first adaptor at one end and a second adaptor at the other end. DNA fragments containing both adaptors are then selected. The asymmetrical adaptors described herein allow one to determine the direction of the polynucleotides, which will, among other things, simplify the process of sequence analyses. In some embodiments, one of the adaptors contains a recognition sequence that is complementary to a primer for single-strand polynucleotide amplification, thus allowing simultaneous selection of DNA fragments containing the adaptor and amplification of the selected polynucleotide to produce single-stranded polynucleotides. The single-strand polynucleotide amplification method allows high accuracy amplification of the target DNA. The present application thus provides a simple and elegant method that simultaneously allows efficiency, sensitivity, and accuracy of nucleic acid sequencing.

Thus, in some embodiments, there is provided a method of generating single-stranded polynucleotides comprising asymmetric adaptor sequences, comprising: i) ligating one or more DNA fragments to: a) a first adaptor comprising a tag; and b) a second adaptor comprising a recognition sequence; ii) selecting DNA fragments comprising the first adaptor based on the presence of the tag; and iii) amplifying the DNA fragments selected from step ii) by single-strand polynucleotide amplification using a primer comprising RNA and hybridizing the primer to the recognition sequence, thereby selectively amplifying DNA fragments comprising the second adaptor.

In some embodiments, there is provided a method of generating single-stranded polynucleotides comprising asymmetric adaptor sequences, comprising: i) ligating one or more DNA fragments to: a) a first adaptor comprising a tag; and b) a second adaptor comprising a recognition sequence; ii) selecting DNA fragments comprising the first adaptor based on the presence of the tag; iii) amplifying DNA fragments selected from step ii) using a primer comprising RNA and hybridizing the primer to the recognition sequence, thereby selectively amplifying DNA fragments comprising the second adaptor, wherein the DNA fragments are amplified by: a) extending the primer comprising RNA in a complex comprising the DNA fragment to be amplified and b) cleaving the RNA portion of the primer with an enzyme that cleaves RNA from an RNA/DNA hybrid such that another primer comprising RNA hybridizes to the DNA fragment and repeats primer extension by strand displacement.

In some embodiments, there is provided a method of generating single-stranded polynucleotides comprising asymmetric adaptor sequences, comprising: i) ligating one or more DNA fragments to: a) a first adaptor comprising a tag; and b) a second adaptor comprising a recognition sequence; ii) selecting DNA fragments comprising the first adaptor based on the presence of the tag; iii) annealing a primer comprising RNA to the recognition sequence on the selected DNA fragment comprising the second adaptor, iv) extending the primer comprising RNA in a complex comprising the DNA fragment to be amplified, and v) cleaving the RNA portion of the primer with an enzyme that cleaves RNA from an RNA/DNA hybrid such that another primer comprising RNA hybridizes to the DNA fragment and repeats primer extension by strand displacement.

In some embodiments, the one or more DNA fragments are generated from a double-stranded target DNA. The double-stranded target DNA can be genomic DNA, DNA produced by primer extension reaction, cDNA, mitochondrial DNA, chloroplast DNA, plasmid DNA, bacterial artificial chromosomes, yeast artificial chromosomes, or a combination thereof.

In some embodiments, the double-stranded target DNA is present in a sample. In some embodiments, the sample is a tissue sample. In some embodiments, the sample is a body fluid sample. In some embodiments, the sample is a tumor sample. In some embodiments, the sample is obtained from an individual having cancer. In some embodiments, the sample is processed prior to the generation of the DNA fragments for the methods described herein. In some embodiments, the sample is used directly to generate the DNA fragments for the methods described herein.

In some embodiments, the sample is a tissue sample. In some embodiments, the sample is polynucleotides extracted from a tissue sample. In some embodiments, the sample is a single cell. In some embodiments, the sample is polynucleotides extracted from a single cell.

In some embodiments, the double-stranded target DNA is present in the sample at an amount of no more than about 500 ng. In some embodiments, each sample comprises at least about 1 pg, 10 pg, 100 pg, 1 ng, 10 ng, 20 ng, 30 ng, 40 ng, 50 ng, 60 ng, 75 ng, 100 ng, 150 ng, 200 ng, 250 ng, 300 ng, 400 ng, 500 ng, 1 μg, 1.5 μg, 2 μg, or more polynucleotide material. In some embodiments, the sample comprises no more than about 1 pg, 10 pg, 100 pg, 1 ng, 10 ng, 20 ng, 30 ng, 40 ng, 50 ng, 60 ng, 75 ng, 100 ng, 150 ng, 200 ng, 250 ng, 300 ng, 400 ng, 500 ng, 1 μg, 1.5 μg, or 2 μg polynucleotide material.

The DNA fragments can be generated in a many ways. For example, the double-stranded target DNA can be fragmented by acoustic sonication, and/or treatment with one or more enzymes under conditions suitable for the one or more enzymes to generate random double-stranded nucleic acid breaks (which can include DNase I, Fragmentase, and variants thereof). In some embodiments, the fragmentation comprises treating the double-stranded target DNA with one or more restriction endonucleases. The fragments generated can have an average length of about 50 to about 10,000 nucleotides, such as an average length of about 100 to about 10,000 nucleotides, or about 500 to about 25,000 nucleotides.

The adaptors described herein can be single-stranded, double-stranded, or partial duplex. In general, a partial duplex adapter comprises one or more single-stranded regions and one or more double-stranded regions. Double-stranded adaptors can comprise two separate oligonucleotides hybridized to one another, and hybridization may leave one or more blunt ends, one or more 3′ overhangs, one or more 5′ overhangs, one or more bulges resulting from mismatched and/or unpaired nucleotides, or any combinations thereof. In some embodiments, a single-stranded adaptor comprises two or more sequences that are able to hybridize with one another. When two such hybridizable sequences are contained in a single-stranded adaptor, hybridization yields a hairpin structure (hairpin adaptor). When the two hybridized regions are separated from one another by a non-hybridizable region, a “bubble” results.

Methods for ligating two polynucleotides are known in the art, and include without limitation, enzymatic and non-enzymatic (e.g., chemical) methods. Examples of ligation reactions that are non-enzymatic include the non-enzymatic ligation techniques described in U.S. Pat. Nos. 5,780,613 and 5,746,930. In some embodiments, the adaptors are ligated to the polynucleotide fragments by a ligase, for example a DNA ligase or RNA ligase. Multiple ligases, each having characterized reaction conditions, are known in the art, and include, without limitation, NDA+-dependent ligases including tRNA ligase, Taq DNA ligase, ATP-dependent ligases such as T4 RNA ligase, T4 DNA ligase, T3 DNA ligase, T7 DNA ligase, Pfu DNA ligase, DNA ligase I, DNA ligase III, DNA ligase IV, and genetically engineered variants thereof. Ligation can be between polynucleotides having complementary overhangs, or between two blunt ends. Generally, a 5′ phosphate is utilized in a ligation reaction. The 5′ phosphate can be provided by the polynucleotide fragment, the adaptors, or both. 5′ phosphate can be added or removed from the polynucleotides to be ligated, as needed.

In addition to the tag and the recognition sequences described further below in detail, the first and second adaptors may further comprise one or more nucleic acid binding sites (for example for attachment to a sequencing platform, such as a flow cell for massive parallel sequencing, such as developed by IIlumina, Inc.), one or more random or near-random sequences (for example one or more nucleotides selected at random from a set of two or more different nucleotides at one or more positions), or combinations thereof.

The present methods use a first adaptor comprising a tag. The tag allows the nucleic acid comprising the first adaptor to be recognized and separated from nucleic acid not containing the first adaptor. In certain cases, the tag specifically binds to a ligand thereby facilitating the separation of the molecule to which the tag is attached from other molecules that do not contain the tag. Exemplary pairs of tag/ligands include, but are not limited to, antibody/antigen, antigen/antibody, avidin/biotin, biotin/avidin, streptavidin/biotin, biotin/streptavidin, glutathione/GST, GST/glutathione, maltose binding protein/amylose, amylose/maltose binding protein, cellulose binding protein and cellulose, cellulose/cellulose binding protein, etc. In some embodiments, the tag is an epitope for an antibody, for example a his tag or a FLAG tag. In some embodiments, the tag is biotin, and the nucleic acid sequence comprising biotin can be selected by using its ligand avidin or streptavidin.

In some embodiments, the tag is a nucleic acid tag sequence that distinguishes it from other nucleic acid sequences, and the polynucleotide having the first adaptor (which contains the nucleic acid tag sequence) can be selected by using a nucleic acid that is complementary to the nucleic acid tag sequence.

The tag can be conjugated to the first adaptor, or, when the tag is a nucleic acid tag sequence, it can be part of the nucleic acid sequence of the first adaptor. When the tag is a molecule conjugated to the first adaptor, the tag molecule can be conjugated to any nucleic acid residue on the first adaptor, either directly or indirectly. For example, in some embodiments, the tag is conjugated to the 5′ end of one strand of the first adaptor. In some embodiments, the tag is conjugated to the 3′ end of one strand of the first adaptor. In some embodiments, the tag is conjugated to an internal nucleic acid residue of the first adaptor. In some embodiments, the tag is cleavable from the nucleic acid residue such that it can be removed after the separation steps.

When the tag is a nucleic acid tag sequence, it can be present at the 5′ end, the 3′ end, or in the internal region of the first adaptor nucleic acid sequence.

In some embodiments, the ligand recognizing the tag is used to select for the tag-containing polynucleotides. The ligand can be coupled (either directly or indirectly) to a supporting material, which in turn provides a physical or chemical means of separating the tag-containing polynucleotides recognized by the ligand.

In some embodiments, the supporting material is a solid support. For example, the ligand can be coupled, either directly or indirectly, to plates, tubes, bottles, flasks, magnetic beads, magnetic sheets, porous matrices, or any solid surfaces and the like. Agents or molecules that may be used to link the ligand to the solid support include, but are not limited to, lectins, avidin/biotin, inorganic or organic linking molecules. The physical separation can be effected, for example, by filtration, isolation, magnetic field, centrifugation, washing, etc.

In some embodiments, the solid support is a bead, a membrane, a cartridge, a filter, a microtiter plate, a test tube, solid powder, a cast or extrusion molded module, a mesh, a fiber, a magnetic particle composite, or any other solid materials. The solid support may be coated with a substance such as polyethylene, polypropylene, poly(4-methulbutene), polystyrene, polyacrylate, polyethylene terephthalate, rayon, nylon, poly(vinyl butyrate), polyvinylidene difluoride (PCDF), silicones, polyformaldehyde, cellulose, cellulose acetate, nitrocellulose, and the like. In some embodiments, the solid support may be coated with a ligand or impregnated with the ligand.

Other solid support that can be used in the methods described herein include, but are not limited to, gelatin, glass, sepharose macrobeads, dextran microcarriers such as CYTODES® (Pharmacia, Uppsala, Sweden). Also contemplated are polysaccharide such as agrose, alginate, carrageenan, chitin, cellulose, dextran or starch, polyacrylamide, polystyrene, polyacrolein, polyvinyl alcohol, polymethylacrylate, perfluorocarbon, inorganic compounds such as silica, glass, kieselquhr, alumina, iron oxide or other metal oxides, or copolymers consisting of any combination of two or more naturally occurring polymers, synthetic polymers or inorganic compounds. In some embodiments, the solid support is a column (such as a Sepharose column).

Once nucleic acid sequences comprising the first adaptor comprising the tag are selected, they can be subjected to single-strand polynucleotide amplification as described below using a primer comprising an RNA portion that hybridizes to the recognition sequence on the second adaptor. Because only nucleic acid sequences comprising the second adaptor will be amplified, the amplification step also constitutes a second selection step that allows selection of polynucleotides containing both the first adaptor and the second adaptor.

In some embodiments, one strand of the DNA fragment is physically separated from its complementary strand before it is used as a template for the single-strand polynucleotide amplification. For example, when the ligand for the tag is immobilized on a solid support, the bound nucleic acid can be denatured, and the complementary strand not comprising the tag can be eluted from the solid support. The eluted strand, which contains the sequence of the first adaptor but not the tag, can then be subject to single-strand polynucleotide amplification methods. Alternatively, the nucleic acid strand bound to the solid support can be subjected to single-strand polynucleotide amplification method.

The second adaptor comprises a recognition sequence which can be used for primer hybridization, which in turn is required for single-strand polynucleotide amplification. The recognition sequence are typically, but not necessarily, about 5 to about 200 nucleotides long, including for example about 5 to about 10, about 10 to about 15, about 15 to about 20, about 20 to about 25, about 25 to about 30, about 30 to about 35, about 35 to about 40, about 40 to about 45, or about 45 to about 50, about 50 to about 100, about 100 to about 200 nucleotides long. The primer in some embodiments is an RNA primer. In some embodiments, the primer is an RNA/DNA composite primer, and the RNA portion of the RNA/DNA chimer primer can be any of 30%, 40%, 50%, 60%, 70%, 80%, 90%, or more of the entire length of the primer.

The present application in some embodiments also provides a method of preparing single-stranded polynucleotides by using adaptors having 5′ or 3′ overhang. Double-stranded target DNA cleaved with a restriction endonuclease creates a 5′ or 3′ overhang. Adaptors having a 5′ or 3′ overhang that is complementary to the 5′ or 3′ overhang can therefore be selectively ligated to one end of the DNA fragment, allowing directional amplification of the DNA fragment by using a primer that hybridizes to a recognition sequence on the adaptor.

Thus, in some embodiments, there is provided a method of generating single-stranded polynucleotides comprising an adaptor sequence from a double-stranded target DNA, comprising: i) cleaving the double-stranded target DNA with a restriction endonuclease to generate DNA fragments having a 5′ or 3′ overhang; ii) ligating the DNA fragments with an adaptor that comprises a) a single-stranded 5′ or 3′ overhang complementary to the 5′ or 3′ overhang of the DNA fragments and b) a recognition sequence; and iii) amplifying the DNA fragments ligated to the adaptor by single-strand polynucleotide amplification using a primer comprising RNA and hybridizing the primer to the recognition sequence.

In some embodiments, there is provided a method of generating single-stranded polynucleotides comprising an adaptor sequence from a double-stranded target DNA, comprising: i) cleaving the double-stranded target DNA with a restriction endonuclease to generate DNA fragments having a 5′ or 3′ overhang; ii) ligating the DNA fragments with an adaptor that comprises a) a single-stranded 5′ or 3′ overhang complementary to the 5′ or 3′ overhang of the DNA fragments and b) a recognition sequence; and iii) amplifying the DNA fragments ligated to the adaptor using a primer comprising RNA and hybridizing the primer to the recognition sequence, and wherein the DNA fragments are amplified by: a) extending the primer comprising RNA in a complex comprising the DNA fragment to be amplified and b) cleaving the RNA portion of the primer with an enzyme that cleaves RNA from an RNA/DNA hybrid such that another primer comprising RNA hybridizes to the DNA fragment and repeats primer extension by strand displacement.

In some embodiments, there is provided a method of generating single-stranded polynucleotides comprising an adaptor sequence from a double-stranded target DNA, comprising: i) cleaving the double-stranded target DNA with a restriction endonuclease to generate DNA fragments having a 5′ or 3′ overhang; ii) ligating the DNA fragments with an adaptor that comprises a) a single-stranded 5′ or 3′ overhang complementary to the 5′ or 3′ overhang of the DNA fragments and b) a recognition sequence; iii) annealing a primer comprising RNA to the recognition sequence on the DNA fragment comprising the adaptor, iv) extending the primer comprising RNA in a complex comprising the DNA fragment to be amplified, and v) cleaving the RNA portion of the primer with an enzyme that cleaves RNA from an RNA/DNA hybrid such that another primer comprising RNA hybridizes to the DNA fragment and repeats primer extension by strand displacement.

In some embodiments, the restriction sites are pre-selected. By carefully examining the restriction sites on the target DNA and carefully choosing the restriction endonuclease, one would be able to selectively amplify one strand of a DNA fragment. Subsequent to the generation of the single-stranded polynucleotides, the polynucleotides of interest can be further enriched by using probes carefully chosen to pull down the polynucleotides of interest.

Also provided herein are single-stranded polynucleotides generated using the methods described herein. Thus, for example, in some embodiments, there are provided single-stranded polynucleotides comprising: 1) a first adaptor comprising a tag; and 2) a second adaptor comprising a recognition sequence.

In some embodiments, there is also provided a method of generating a library of polynucleotides comprising adapter sequences using the single-stranded polynucleotides generated by the methods described herein.

In some embodiments, there is provided a method of generating an array (such as microarray) using the single-stranded polynucleotides generated by the methods described herein.

### II. Single-Strand Polynucleotide Amplification

The single-strand polynucleotides described herein can be generated from single-stranded or double-stranded DNA or RNA. The methods generally involve use of a primer comprising an RNA portion. In some embodiments, the primer is an RNA primer. In some embodiments, the primer is a DNA/RNA composite primer. Methods of single-strand polynucleotide amplification using DNA/RNA primers are described in U.S. Pat. No. 6,692,918 and further below. Methods of single-strand polynucleotide amplification using an RNA primer is described herein as well as in Provisional Application, Attorney Docket 70178-30003.00, entitled “Single-Strand Polynucleotide Amplification Methods,” filed concurrently with this application and incorporated herein by reference.

Generally, the amplification methods work as follows: a primer comprising RNA is allowed to hybridize to the DNA template. A polymerase (such as DNA polymerase) is used to effect copying of the template sequence by extending the primer. An enzyme which cleaves RNA from an RNA/DNA hybrid (such as RNase H) cleaves (removes) RNA sequence from the hybrid, leaving sequence on the template strand available for binding by another primer. Another strand is produced by the polymerase (such as DNA polymerase), which displaces the previously replicated strand, resulting in displaced extension product.

In some embodiments, the method comprises: a) extending the primer comprising RNA in a complex comprising: i) the DNA fragment to be amplified and ii) the primer comprising RNA, wherein the primer comprising RNA is hybridized to the DNA fragment to be amplified; and b) cleaving the RNA portion of the primer with an enzyme that cleaves RNA from an RNA/DNA hybrid such that another primer comprising RNA hybridizes to the DNA fragment and repeats primer extension by strand displacement; whereby multiple copies of single-stranded polynucleotides are generated.

The total length of the primer (such as the composite primer or the RNA primer) can be from about 10 to about 40 nucleotides, including for example about 15 to about 30 nucleotides, about 20 to about 25 nucleotides. In some embodiments, the length of the primer is at least about any of 10, 15, 20, 25 nucleotides. In some embodiments, the length of the primer is no more than about any of 25, 30, 40, or 50 nucleotides. To achieve hybridization (which, as is well known and understood in the art, depends on other factors such as, for example, ionic strength and temperature), the primers are at least about 60%, 70%, 75%, 80%, 85%, 90%, or 95% complementary to the recognition portion of the second adaptor.

The amplification methods described herein in some embodiments uses a DNA polymerase. In some embodiments, the DNA polymerase is one that is capable of extending a nucleic acid primer along a nucleic acid template that is comprised at least predominantly of deoxyribonucleotides. The polymerase should be able to displace a nucleic acid strand from the polynucleotide to which the displaced strand is bound, and, generally, polymerases exhibiting more strand displacement capability (i.e., compared to other polymerases which do not have as much strand displacement capability) are preferable. In some embodiments, the DNA polymerase has high affinity for binding at the 3′-end of an oligonucleotide hybridized to a nucleic acid strand. In some embodiments, the DNA polymerase does not possess substantial nicking activity. In some embodiments, the polymerase has little or no 5′→3′ exonuclease activity so as to minimize degradation of primer or primer extension polynucleotides. Generally, this exonuclease activity is dependent on factors such as pH, salt concentration, and so forth, all of which are familiar to one skilled in the art. Mutant DNA polymerases in which the 5′→3′ exonuclease activity has been deleted, are known in the art and are suitable for the amplification methods described herein. Suitable DNA polymerases for use in the methods and compositions of the present invention include those disclosed in U.S. Pat. Nos. 5,648,211 and 5,744,312, which include exo-Vent (New England Biolabs), exo-Deep Vent (New England Biolabs), Bst (BioRad), exo-Pfu (Stratagene), Bca (Panvera), sequencing grade Taq (Promega), and thermostable DNA polymerases from thermoanaerobacter thermohydrosulfuricus. In some embodiments, the DNA polymerase displaces primer extension products from the template nucleic acid in at least about 25%, more preferably at least about 50%, even more preferably at least about 75%, and most preferably at least about 90%, of the incidence of contact between the polymerase and the 5′ end of the primer extension product. In some embodiments, the use of thermostable DNA polymerases with strand displacement activity is used. Such polymerases are known in the art, such as described in U.S. Pat. No. 5,744,312 (and references cited therein). Preferably, the DNA polymerase has little to no proofreading activity. In some embodiments, the DNA polymerase is selected from the group consisting of a strand-displacing DNA polymerase, a high fidelity DNA polymerase, a polymerase that has proofreading activity, a T7 DNA polymerase, and an E. coli DNA polymerase I.

The enzyme that cleaves RNA from an RNA/DNA hybrid in some embodiments is a ribonuclease that cleaves ribonucleotides regardless of the identity and type of nucleotides adjacent to the ribonucleotide to be cleaved. In some embodiments, the enzyme cleaves independent of sequence identity. Examples of suitable ribonucleases for the methods and compositions of the present invention are well known in the art, including ribonuclease H (RNase H).

Appropriate reaction components and conditions for carrying out the methods described herein are those that permit nucleic acid amplification. Such components and conditions are known to persons of skill in the art, and are described in various publications, such as U.S. Pat. No. 5,679,512 and PCT Pub. No. WO99/42618. For example, a buffer may be Tris buffer, although other buffers can also be used as long as the buffer components are non-inhibitory to enzyme components of the methods of the invention. The pH can be about 5 to about 11, for example from about 6 to about 10, from about 7 to about 9, from about 7.5 to about 8.5, or about 8.5. The reaction mixture can also include bivalent metal ions such as Mg²⁺ or Mn²⁺, at a final concentration of free ions that is within the range of from about 0.01 to about 10 mM, including for example from about 1 to about 5 mM. The reaction mixture can also include other salts, such as KCl, that contribute to the total ionic strength of the medium. For example, the range of a salt such as KCl is from about 0 to about 100 mM, including from about 0 to about 75 mM, such as from about 0 to about 50 mM. The reaction mixture may also contain a single-stranded DNA binding protein; for example, it may contain 3 ug T4gp32 (USB). The reaction mixture can further include additives that could affect performance of the amplification reactions, but that are not integral to the activity of the enzyme components of the methods. Such additives include proteins such as BSA, and non-ionic detergents such as NP40 or Triton. Additional reagents, such as DTT, that are capable of maintaining enzyme activities can also be included; for example, DTT may be included at a concentration of about 1 to about 5 mM. Such reagents are known in the art.

Where appropriate, an RNase inhibitor (such as Rnasine) that does not inhibit the activity of the RNase employed in the method can also be included. The reaction can occur at a constant temperature or at varying temperatures. In some embodiments, the reactions are performed isothermally, which avoids the cumbersome thermocycling process. The amplification reaction is carried out at a temperature that permits hybridization of the oligonucleotides (primer, TSO, blocker sequence, and/or PTO) of the present invention to the template polynucleotide and that does not substantially inhibit the activity of the enzymes employed. The temperature can be in the range of about 25° C. to about 85° C., including for example about 30° C. to about 75° C., about 37° C. to about 70° C., or about 55° C. In some embodiments, the reaction is carried out at a temperature in the range of about 25° C. to about 85° C., about 30° C. to about 75° C., and about 37° C. to about 70° C.

The reaction mixture containing the primers, probes, and samples may first be denatured by incubation at 95° C. for about 2 to about 5 min, and the primer(s) allowed to anneal to target at 55° C. for about 5 min.

Nucleotide and/or nucleotide analogs, such as deoxyribonucleoside triphosphates, that can be employed for synthesis of the primer extension products in the methods of the invention can be provided in the amount of from about 50 to about 2500 μM, about 100 to about 2000 μM, about 500 to about 1700 μM, or about 800 to about 1500 μM. Deoxyribose nucleoside triphosphates (dNTPs) may be used at a concentration of, for example, about 250 to about 500 uM. In some embodiments, a nucleotide or nucleotide analog whose presence in the primer extension strand enhances displacement of the strand (for example, by causing base pairing that is weaker than conventional AT, CG base pairing) is included. Such nucleotide or nucleotide analogs include deoxyinosine and other modified bases, all of which are known in the art. Nucleotides and/or analogs, such as ribonucleoside triphosphates, that can be employed for synthesis of the RNA transcripts in the methods of the invention are provided in the amount of from about 0.25 to about 6 mM, about 0.5 to about 5 mM, about 0.75 to about 4 mM, or about 1 to about 3 mM.

The oligonucleotide components of the amplification reactions of the invention are generally in excess of the number of target nucleic acid sequence to be amplified. They can be provided at about or at least about any of the following: 10, 10 ², 10⁴, 10⁶, 10⁸, 10¹⁰, 10¹² times the amount of target nucleic acid. The primer (composite primer or RNA primer) can be provided at about or at least about any of the following concentrations: 50 nM, 100 nM, 500 nM, 1000 nM, 2500 nM, 5000 nM.

In one embodiment, the foregoing components are added simultaneously at the initiation of the amplification process. In another embodiment, components are added in any order prior to or after appropriate time points during the amplification process, as required and/or permitted by the amplification reaction. Such time points can be readily identified by a person of skill in the art. The enzymes used for nucleic acid amplification according to the methods of the present invention can be added to the reaction mixture either prior to the nucleic acid denaturation step, following the denaturation step, or following hybridization of the primer to the target DNA, as determined by their thermal stability and/or other considerations known to the person of skill in the art.

The amplification reactions can be stopped at various time points, and resumed at a later time. Said time points can be readily identified by a person of skill in the art. Methods for stopping the reactions are known in the art, including, for example, cooling the reaction mixture to a temperature that inhibits enzyme activity. Methods for resuming the reactions are also known in the art, including, for example, raising the temperature of the reaction mixture to a temperature that permits enzyme activity. In some embodiments, one or more of the components of the reactions is replenished prior to, at, or following the resumption of the reactions. Alternatively, the reaction can be allowed to proceed (i.e., from start to finish) without interruption.

### III. Methods of Enriching Polynucleotides of Interest

The present application provides methods of analyzing target nucleotides, including RNA (such as double-stranded RNA and single-stranded RNA) and DNA (such as double-stranded DNA, for example genomic DNA). The methods generally involve contacting a population of single-stranded polynucleotides amplified from said target polynucleotides (for example by using the single-strand polynucleotide amplification methods described above) with a set of probes, thereby enriching polynucleotides containing one or more regions that are hybridizable to the probes. The enrichment methods described herein reduce the complexity of the polynucleotide sequences to be analyzed and allow the polynucleotides of interest to be better represented in the pool.

Thus, in some embodiments, the method comprises: 1) contacting a population of single-stranded polynucleotides generated from a target polynucleotide with a set of probes that are hybridizable to one or more regions on the target polynucleotides; and 2) separating polynucleotides that are bound to the probes from the rest of the polynucleotides, wherein polynucleotides comprising the one or more desired regions are enriched.

In some embodiments, the population of single-stranded polynucleotides is generated from the target polynucleotide by a single-strand polynucleotide amplification method using a primer comprising RNA. In some embodiments, the population of single-stranded polynucleotides comprises one or more adaptor sequence and are generated, for example, using one of the methods described herein for generating single-stranded polynucleotides comprising adaptor sequence(s).

The probes used herein can be hybridizable to any regions of interest. In some embodiments, the one or more desired regions are regions where oncogenes are located. In some embodiments, the one or more desired regions are regions wherein one or more mutations are located. In some embodiments, the one or more desired regions are regions where one or more polymorphisms are located.

The number of probes may be selected based on the complexity level of the sample material and the length of the polynucleotide that is desirably sequenced. The methods described herein may be done using a single oligonucleotide or a plurality (i.e., a mixture of at least 2, at least 5, at least 10, at least 50, at least 100, at least 500, at least 1000, at least 10,000, at least 100,000, or more) of different oligonucleotides. These oligonucleotides can be used to enrich for a plurality (i.e., at least 2, at least 5, at least 10, at least 50, at least 100, at least 500, at least 1000, at least 10,000, at least 100,000, or more) different regions on the polynucleotide sequence.

The probes used in the methods described herein can be of any length, including, but not limited to, about 200 to about 500, about 500 to about 1,000, about 1,000 to about 2,000, about 2,000 to about 5,000, about 5,000 to about 10,000, about 10,000 to about 20,000 nucleotides long. The probes in some embodiments are provided in access to the polynucleotides to be enriched. For example, in some embodiments, the probes are at least about any of 10, 10², 10³, 10⁴, or more times the amount of the polynucleotides to be enriched. In some embodiments, the probes are no more than about 10, 10², 10³, or 10⁴ times the amount of the polynucleotides to be enriched.

The level of complexity reduction obtained by the enrichment method may enable reduction of 10%, 20%, 30%, 40%, 50%, 60%, 70%, 80%, 90%, 95%, or 99% of the complexity of the initial polynucleotide pool, or may involve selection of only a few percent of the polynucleotides, or even a few thousand base pairs. For example, when the initial polynucleotide pool is generated from a genomic DNA, the complexity of the polynucleotides may be reduced from 3 billion base pairs to 10 million base pairs or less, depending on the size of the initial genome and the level of reduction required. Using this method, highly repetitive DNA sequences which comprise, for example 40% of the human genomic DNA, can be removed quickly and efficiently from a complex population.

### IV. Methods of Analyzing Polynucleotides

The polynucleotides generated using the methods described herein (such as single-stranded polynucleotides comprising adaptor(s) and polynucleotides enriched by probes) can be further subject to analysis. The analyses can include, but are not limited to, polynucleotide sequencing, mutation analysis, determination of polymorphism, etc. The methods described herein are particularly useful for identifying mutations in a polynucleotide sample, predicting responsiveness of an individual to a drug; predicting pharmacokinetics of drug in an individual, predicting therapeutic outcome of a treatment in an individual. The methods can also be useful for genetic testing such as genetic testing for prenatal screening.

The polynucleotides can be analyzed by any analysis methods, including, but not limited to, DNA sequencing (using Sanger, pyrosequencing or the sequencing systems of Roche/454, Helicos, Illumina/Solexa, and ABI (SOLID)), a polymerase chain reaction assay, a bead array assay, a primer extension assay, an enzyme mismatch cleavage assay, a branched hybridization assay, a NASBA assay, a molecular beacon assay, a cycling probe assay, a ligase chain reaction assay, an invasive cleavage structure assay, an ARMS assay, or a sandwich hybridization assay, for example. The polynucleotide molecules can be sequenced or analyzed for the presence of SNPs or other differences relative to a reference sequence.

In some embodiments, the polynucleotides generated by the methods described herein can be used for NP haplotyping of a chromosomal region that contains two or more SNPS, for enriching for DNA sequences for paired-end sequencing methods, for generating target fragments for long-read sequences, isolating inversion, deletion, and translocation breakpoints, for sequencing entire gene regions (exons and introns) to uncover mutations causing aberrant splicing or regulation, and for the production of long probes for chromosome imaging, e.g., Bionanomatrix, optical mapping, or fiber-FISH-based methods.

Polymorphisms, particularly single nucleotide polymorphism (“SNP”) are essentially randomly distributed throughout the genome. A polymorphism may be an insertion, deletion, duplication, or rearrangement of any length of a sequence, including single nucleotide deletions, insertions, or base change. The polymorphism may be naturally occurring, or it may be associated with variant phenotypes. The use of the methods described herein, for example through the enrichment of the sequences of interest, allows substantially reproducible access to substantially similar reduced-complexity subpopulations in different individuals in a population or even in different samples from a single individual. Because polymorphisms are essentially randomly distributed throughout the genome, a number of polymorphic sequences will be present in the reduced-complexity population of nucleic acid sequences. Such reduced-complexity subpopulation can be analyzed to either identify polymorphisms or to determine the genotype of polymorphic loci within that sub-population.

The methods described herein can also be useful, for example, in the field of pharmacogenomics, which seeks to correlate the knowledge of specific alleles of polymorphic loci with the way in which individuals in a population respond to particular drug. A broad estimate is that, for every drug, between 10% and 40% of individuals do not respond optimally. In order to create a response profile for a given drug, the genotype with regard to polymorphic loci of those individuals receiving the drug must be correlated with the therapeutic outcome of the drug. This is frequently performed with analysis of a large number of polymorphic loci. Once a genetic drug response profile has been estimated by analysis of polymorphic loci in a population, a clinical patient's genotype with respect to those loci related to responses to particular drugs must be determined. Therefore, the ability to identify the sequence of a large number of polymorphic loci in a large number of individuals is important for both establishment of a drug response profile and for identification of an individual's genotype for clinical applications.

The polynucleotides generated using the methods described herein (such as single-stranded polynucleotides comprising adaptor(s) and polynucleotides enriched by probes) are subjected to sequencing analysis using the Illumina sequencing method. The Illumina sequencing method includes bridge amplification technology, in which primers bound to a solid phase are used in the extension and amplification of solution phase single-stranded nucleic acid acids prior to SBS. (See, e.g., Mercier, et al. (2005) “Solid Phase DNA Amplification: A Brownian Dynamics Study of Crowding Effects.” Biophysical Journal 89: 32-42; Bing, et al. (1996) “Bridge Amplification: A Solid Phase PCR System for the Amplification and Detection of Allelic Differences in Single Copy Genes.” Proceedings of the Seventh International Symposium on Human Identification, Promega Corporation Madison, Wis.)

Illumina sequencing technology entails preparing single-stranded nucleic acids flanked with paired-end adapter sequences. Each of the paired-end adapters contains a unique primer hybridization sequence. The nucleic acids are distributed on to a flow cell surface that is coated with single-stranded oligonucleotides that correspond to the primer hybridization sequences present on the adapters flanking the single-stranded nucleic acids. The single-stranded, adapter-ligated nucleic acids are bound to the surface of the flow cell and exposed to reagents for polymerase-based extension. Priming occurs as the free/distal end of a ligated fragment “bridges” to a complementary oligonucleotide on the surface, and during the annealing step, the extension product from one bound primer forms a second bridge strand to the other bound primer. Repeated denaturation and extension results in localized amplification of single molecules in millions of unique locations, creating clonal “clusters” across the flow cell surface.

The flow cell is then placed in a fluidics cassette within a sequencing module, where primers, DNA polymerase, and fluorescently-labeled, reversibly terminated nucleotides, e.g., A, C, G, and T, are added to permit the incorporation of a single nucleotide into each clonal DNA in each cluster. Each incorporation step is followed by the high-resolution imaging of the entire flow cell to identify the nucleotides that were incorporated at each cluster location on the flow cell. After the imaging step, a chemical step is performed to deblock the 3′ ends of the incorporated nucleotides to permit the subsequent incorporation of another nucleotide. Iterative cycles are performed to generate a series of images each representing a single base extension at a specific cluster. This system typically produces sequence reads of up to 20-50 nucleotides. Further details regarding this sequencing system are discussed in, e.g., Bennett, et al. (2005) “Toward the 1,000 dollars human genome.” Pharmacogenomics 6: 373-382; Bennett, S. (2004) “Solexa Ltd.” Pharmacogenomics 5: 433-438; and Bentley, D. R. (2006) “Whole genome re-sequencing.” Curr Opin Genet Dev 16: 545-52.

The first stage in preparing template for the Illumina system is DNA fragmentation by nebulization. However, the wide size distribution of generated fragments is uneconomical, as the 20-200 fragments that can be used in subsequent template preparation steps represent approximately 10% of the total DNA after nebulization. Moreover, approximately half of the DNA vaporizes after nebulization, meaning that only 5% of the original DNA is used to prepare sequencing template. Additionally, 50% of the DNA strands in the clonal clusters that are formed during bridge amplification, as strands with free 5′ ends are removed prior to the sequencing reaction.

The methods provided herein can be readily adapted for use with the Illumina platform. Specifically, the adaptor sequences described herein are ideally suited for the purpose of the Illumina sequencing methods.

In some embodiments, the polynucleotides generated by the methods described herein are analyzed using single-molecule real-time sequencing. Single molecule real-time sequencing (SMRT) is another massively parallel sequencing technology that can be used to sequence circularized single-stranded nucleic acids in a high-throughput manner. Developed and commercialized by Pacific Biosciences, SMRT technology relies on arrays of multiplexed zero-mode waveguides (ZMWs) in which, e.g., thousands of sequencing reactions can take place simultaneously. The ZMW is a structure that creates an illuminated observation volume that is small enough to observe, e.g., the template-dependent synthesis of a single-stranded DNA molecule by a single DNA polymerase (See, e.g., Levene, et al. (2003) “Zero Mode Waveguides for Single Molecule Analysis at High Concentrations,” Science 299: 682-686). When a DNA polymerase incorporates complementary, fluorescently labeled nucleotides into the DNA strand that is being synthesized, the enzyme holds each nucleotide within the detection volume for tens of milliseconds, e.g., orders of magnitude longer than the amount of time it takes an unincorporated nucleotide to diffuse in and out of the detection volume. During this time, the fluorophore emits fluorescent light whose color corresponds to the nucleotide base's identity. Then, as part of the nucleotide incorporation cycle, the polymerase cleaves the bond that previously held the fluorophore in place and the dye diffuses out of the detection volume. Following incorporation, the signal immediately returns to baseline and the process repeats. Additional descriptions of ZMWs and their application in single molecule analyses, such as SMRT sequencing can be found in, e.g., Published U.S. Patent Application No. 2003/0044781, and U.S. Pat. No. 6,917,726, each of which is incorporated herein by reference in its entirety for all purposes. See also, Levene et al. (2003) “Zero Mode Waveguides for single Molecule Analysis at High Concentrations,” Science 299:682-686 and Eid, et al. (2009) “Real-Time DNA Sequencing from Single Polymerase Molecules.” Science 323:133-138.

The polynucleotides generated by the methods described herein can be adapted for use with the SMRT sequencing platform. For example, following synthesis, the single-stranded polynucleotides can be circularized using an enzyme that catalyzes the intramolecular ligation of single-stranded DNA fragments, e.g., CircLigase™, CircLigase™ II, or ThermoPhage™, and distributed to ZMWs. Alternatively, the daughter strands can be fragmented prior to circularization. Optionally, sequences of interest can be enriched from a population of fragmented daughter strands, e.g., as described above, prior to circularization.

In some embodiments, the analysis comprises mutational analysis, including for example mutation analysis can be carried out by any methods known in the art, including DNA sequencing, denaturing HPLC, electrophoresis detection, and conformational difference studies.

### IV. Methods of the Present Application

The present application in one aspect provides a method of analyzing one or more desired regions on a target polynucleotide, wherein the one or more desired regions are hybridizable to a set of probes, comprising: 1) contacting a population of single-stranded polynucleotides generated from said target polynucleotide with the set of probes; 2) separating polynucleotides that are bound to the probes from the rest of the polynucleotides, wherein polynucleotides comprising the one or more desired regions are enriched; and 3) analyzing the separated polynucleotides.

In some embodiments, there is provided a method of analyzing one or more desired regions on a target polynucleotide, wherein the one or more desired regions are hybridizable to a set of probes, comprising: 1) amplifying the target polynucleotide by single-strand polynucleotide amplification to generate a population of single-stranded polynucleotides, 2) contacting the population of single-stranded polynucleotides with the set of probes; 3) separating polynucleotides that are bound to the probes from the rest of the polynucleotides, wherein polynucleotides comprising the one or more desired regions are enriched; and 4) analyzing the separated polynucleotides.

In some embodiments, there is provided a method of analyzing one or more desired regions on a target polynucleotide, wherein the one or more desired regions are hybridizable to a set of probes, comprising: 1) amplifying the target polynucleotide by single-strand polynucleotide amplification using a primer comprising RNA to generate a population of single-stranded polynucleotides, 2) contacting the population of single-stranded polynucleotides with the set of probes; 3) separating polynucleotides that are bound to the probes from the rest of the polynucleotides, wherein polynucleotides comprising the one or more desired regions are enriched; and 4) analyzing the separated polynucleotides.

In some embodiments, there is provided a method of analyzing one or more desired regions on a target polynucleotide, wherein the one or more desired regions are hybridizable to a set of probes, comprising: 1) extending a primer comprising RNA in a complex comprising the target polynucleotide and the primer comprising RNA, wherein the primer comprising RNA is hybridized to the target polynucleotide, 2) cleaving the RNA portion of the primer with an enzyme that cleaves RNA from an RNA/DNA hybrid such that another primer comprising RNA hybridizes to the DNA fragment and repeats primer extension by strand displacement, whereby a population of single-stranded polynucleotides are generated; 3) contacting the population of single-stranded polynucleotides with the set of probes; 4) separating polynucleotides that are bound to the probes from the rest of the polynucleotides, wherein polynucleotides comprising the one or more desired regions are enriched; and 4) analyzing the separated polynucleotides.

In some embodiments, the target polynucleotide is double-stranded DNA (such as genomic DNA). For example, in some embodiments, there is provided a method of analyzing the sequence of one or more desired regions on the genomic DNA of an individual, comprising: 1) fragmenting the genomic DNA to generate DNA fragments; 2) ligating the DNA fragments with an first adaptor comprising a tag and a second adaptor comprising a recognition sequence; 3) subjecting the DNA fragments to a selection process that allows selection of DNA fragments comprising the first adaptor based on the presence of the tag; 4) amplifying the DNA fragments comprising the first adaptor by single-strand polynucleotide amplification using a primer comprising RNA and hybridizing to the recognition sequence, whereby a population of single-stranded polynucleotides are generated; 5) contacting the population of single-stranded polynucleotides with a set of probes hybridizable to the one or more desired regions; 6) separating polynucleotides that are bound to the probes from the rest of the single-stranded polynucleotides, wherein polynucleotides comprising the one or more desired regions are enriched; and 7) analyzing the sequence of the separated polynucleotide molecules.

In some embodiments, there is provided a method of analyzing the sequence of one or more desired regions on the genomic DNA of an individual, comprising: 1) cleaving the genomic DNA with a restriction endonuclease to generate DNA fragments having a 5′ or 3′ overhang; 2) ligating the DNA fragments with an adaptor that comprises a) a sequence complementary to the 5′ or 3′ overhang and b) a recognition sequence; 3) amplifying one strand of the DNA using a primer comprising RNA portion and hybridizing the primer to the recognition sequence (for example by single-strand polynucleotide amplification), whereby a population of single-stranded polynucleotides are generated; 4) contacting the population of stranded polynucleotides with a set of probes hybridizable to the one or more desired regions; 5) separating polynucleotides that are bound to the probes from the rest of the single-stranded polynucleotides, wherein polynucleotides comprising the one or more desired regions are enriched; and 6) analyzing the sequence of the separated polynucleotide molecules.

In some embodiments, there is provided a method of analyzing a double-stranded DNA (such as genomic DNA), comprising i) fragmenting the double-stranded DNA to generate DNA fragments, ii) ligating one or more DNA fragments to: a) a first adaptor comprising a tag; and b) a second adaptor comprising a recognition sequence; iii) selecting DNA fragments comprising the first adaptor based on the presence of the tag; iv) amplifying the DNA fragments selected from step iii) by single-strand polynucleotide amplification using a primer comprising RNA and hybridizing the primer to the recognition sequence, thereby selectively amplifying DNA fragments comprising the second adaptor, whereby a population of single-stranded polynucleotides are generated, and v) analyzing the single-stranded polynucleotides.

In some embodiments, there is provided a method of analyzing a double-stranded DNA (such as genomic DNA), comprising i) fragmenting the double-stranded DNA to generate DNA fragments, ii) ligating one or more DNA fragments to: a) a first adaptor comprising a tag; and b) a second adaptor comprising a recognition sequence; iii) selecting DNA fragments comprising the first adaptor based on the presence of the tag; iv) amplifying the DNA fragments selected from step iii) by single-strand polynucleotide amplification using a primer comprising RNA and hybridizing the primer to the recognition sequence, thereby selectively amplifying DNA fragments comprising the second adaptor, whereby a population of single-stranded polynucleotides are generated, and v) analyzing the single-stranded polynucleotides, wherein the DNA fragments are amplified by: a) extending the primer comprising RNA in a complex comprising the DNA fragment to be amplified (for example by using DNA polymerase) and b) cleaving the RNA portion of the primer with an enzyme that cleaves RNA from an RNA/DNA hybrid (such as RNase H) such that another primer comprising RNA hybridizes to the DNA fragment and repeats primer extension by strand displacement.

In some embodiments, there is provided a method of analyzing a double-stranded DNA (such as genomic DNA), comprising i) fragmenting the double-stranded DNA to generate DNA fragments, ii) ligating one or more DNA fragments to: a) a first adaptor comprising a tag; and b) a second adaptor comprising a recognition sequence; iii) selecting DNA fragments comprising the first adaptor based on the presence of the tag; iii) annealing a primer comprising RNA to the recognition sequence to the selected DNA fragment comprising the second adaptor, iv) extending the primer comprising RNA in a complex comprising the DNA fragment to be amplified, v) cleaving the RNA portion of the primer with an enzyme that cleaves RNA from an RNA/DNA hybrid such that another primer comprising RNA hybridizes to the DNA fragment and repeats primer extension by strand displacement, whereby a population of single-stranded polynucleotides are generated, and vi) analyzing the single-stranded polynucleotides.

In some embodiments, there is provided a method of analyzing a double-stranded DNA (such as genomic DNA), comprising i) fragmenting the double-stranded DNA to generate DNA fragments, ii) ligating one or more DNA fragments to: a) a first adaptor comprising a tag; and b) a second adaptor comprising a recognition sequence; iii) selecting DNA fragments comprising the first adaptor based on the presence of the tag; iii) annealing a primer comprising RNA to the recognition sequence to the selected DNA fragment comprising the second adaptor, iv) extending the primer comprising RNA in a complex comprising the DNA fragment to be amplified, v) cleaving the RNA portion of the primer with an enzyme that cleaves RNA from an RNA/DNA hybrid such that another primer comprising RNA hybridizes to the DNA fragment and repeats primer extension by strand displacement, whereby a population of single-stranded polynucleotides are generated, vi) contacting the population of single-stranded polynucleotides with a set of probes; vii) separating polynucleotides that are bound to the probes from the rest of the polynucleotides, and viii) analyzing the separated polynucleotides.

In some embodiments, there is provided a method of analyzing a double-stranded DNA (such as genomic DNA), comprising: i) cleaving the double-stranded target DNA with a restriction endonuclease to generate DNA fragments having a 5′ or 3′ overhang; ii) ligating the DNA fragments with an adaptor that comprises a) a single-stranded 5′ or 3′ overhang complementary to the 5′ or 3′ overhang of the DNA fragments and b) a recognition sequence; and iii) amplifying the DNA fragments ligated to the adaptor by single-strand polynucleotide amplification using a primer comprising RNA and hybridizing the primer to the recognition sequence, whereby a population of single-stranded polynucleotides are generated, and v) analyzing the single-stranded polynucleotides.

In some embodiments, there is provided a method of analyzing a double-stranded DNA (such as genomic DNA), comprising i) cleaving the double-stranded target DNA with a restriction endonuclease to generate DNA fragments having a 5′ or 3′ overhang; ii) ligating the DNA fragments with an adaptor that comprises a) a single-stranded 5′ or 3′ overhang complementary to the 5′ or 3′ overhang of the DNA fragments and b) a recognition sequence; and iii) amplifying the DNA fragments ligated to the adaptor by single-strand polynucleotide amplification using a primer comprising RNA and hybridizing the primer to the recognition sequence, whereby a population of single-stranded polynucleotides are generated, and v) analyzing the single-stranded polynucleotides, wherein the DNA fragments are amplified by wherein the DNA fragments are amplified by: a) extending the primer comprising RNA in a complex comprising the DNA fragment to be amplified (such as by DNA polymerase) and b) cleaving the RNA portion of the primer with an enzyme that cleaves RNA from an RNA/DNA hybrid (such as RNase H) such that another primer comprising RNA hybridizes to the DNA fragment and repeats primer extension by strand displacement.

In some embodiments, there is provided a method of analyzing a double-stranded DNA (such as genomic DNA), comprising i) cleaving the double-stranded target DNA with a restriction endonuclease to generate DNA fragments having a 5′ or 3′ overhang; ii) ligating the DNA fragments with an adaptor that comprises a) a single-stranded 5′ or 3′ overhang complementary to the 5′ or 3′ overhang of the DNA fragments and b) a recognition sequence; iii) annealing a primer comprising RNA to the recognition sequence to the DNA fragment comprising the adaptor, iv) extending the primer comprising RNA in a complex comprising the DNA fragment to be amplified, v) cleaving the RNA portion of the primer with an enzyme that cleaves RNA from an RNA/DNA hybrid such that another primer comprising RNA hybridizes to the DNA fragment and repeats primer extension by strand displacement, whereby a population of single-stranded polynucleotides are generated, and vi) analyzing the single-stranded polynucleotides.

In some embodiments, there is provided a method of analyzing a double-stranded DNA (such as genomic DNA), comprising i) cleaving the double-stranded target DNA with a restriction endonuclease to generate DNA fragments having a 5′ or 3′overhang; ii) ligating the DNA fragments with an adaptor that comprises a) a single-stranded 5′ or 3′ overhang complementary to the 5′ or 3′ overhang of the DNA fragments and b) a recognition sequence; iii) annealing a primer comprising RNA and hybridizing to the recognition sequence to the DNA fragment comprising the adaptor, iv) extending the primer comprising RNA in a complex comprising the DNA fragment to be amplified, v) cleaving the RNA portion of the primer with an enzyme that cleaves RNA from an RNA/DNA hybrid such that another primer comprising RNA hybridizes to the DNA fragment and repeats primer extension by strand displacement, whereby a population of single-stranded polynucleotides are generated, vi) contacting the population of single-stranded polynucleotides with a set of probes; vii) separating polynucleotides that are bound to the probes from the rest of the polynucleotides, and viii) analyzing the separated polynucleotides.

The polynucleotides to be analyzed by any of the methods described herein can be present in a sample, for example a human sample. In some embodiments, the sample is a tissue sample. In some embodiments, the sample is polynucleotides extracted from a tissue sample. In some embodiments, the sample is a single cell. In some embodiments, the sample is polynucleotides extracted from a single cell.

The methods described herein can also be useful for any one of the polynucleotide analytical methods, including, but not limited to, sequencing a polynucleotide, determining the presence or absence of a mutation in a polynucleotide, analyzing the polymorphism of the polynucleotide.

The methods described herein can be useful for analyzing a polynucleotide sample from an individual, which can be useful for purposes that include, but are not limited to: 1) diagnosing a disease (such as cancer) in an individual, 2) assessing risk of developing a disease (such as cancer) in an individual, 3) determining responsiveness of an individual to a treatment regime (such as cancer treatment), 4) evaluating efficacy of a treatment (such as cancer treatment) on an individual, 5) determining continued treatment (such as cancer treatment) on an individual; and 6) predicting responsiveness of an individual to a treatment regime (such as cancer).

### IV. Kits, Compositions, Reagents, and Article of Manufacture

Also provided herein are kits, reagents, and articles of manufacture useful for the methods described herein.

In some embodiments, there is provided a pair of adaptors comprising a first adaptor comprising a tag and a second adaptor comprising a recognition sequence. In some embodiments, the pair of adaptors is present in the same composition. In some embodiments, the pair of adaptors is present in separate compositions.

In some embodiments, there is provided a composition comprising a plurality of polynucleotide fragments, each polynucleotide fragment comprising a first adaptor at one end and a second adaptor at the second end, wherein the first adaptor comprises a tag, and wherein the second adaptor comprises a recognition sequence. In some embodiments, the polynucleotide fragments in the composition are derived from a different target nucleotide from different samples. Such a composition can be useful, for example, for multiplex polynucleotide sequencing. The polynucleotides can either be the single-stranded polynucleotides described herein, or generated from the single-stranded polynucleotides. In some embodiments, there is provided a library of polynucleotides, wherein each polynucleotides comprise a first adaptor comprising a tag and a second adaptor comprising a recognition sequence. In some embodiments, there is provided an array (such as microarray) of polynucleotides, wherein each polynucleotide comprises a first adaptor comprising a tag and a second adaptor comprising a recognition sequence.

In some embodiments, there is provided a kit useful in the generation of adaptor-containing polynucleotide fragments. In some embodiments, the kit comprises a first adaptor and a second adaptor. In some embodiments, the kit further comprises a primer (such as an RNA primer or a DNA/RNA composite primer). In some embodiments, there is provided a kit comprising: i) a first adaptor comprising a tag; ii) a second adaptor comprising a recognition sequences, and iii) a primer that hybridizes to the recognition sequence (such as a primer comprising RNA, for example an RNA primer or a DNA/RNA composite primer). In some embodiments, the kit further comprises a ligand that binds to the tag. In some embodiments, the kit further comprises a solid support. In some embodiments, the kit further comprises one or more of: 1) a DNA ligase, 2) a DNA polymerase (such as a DNA-dependent DNA polymerase and/or an RNA-dependent DNA polymerase, 3) a DNA endonuclease, 4) a DNA kinase, 5) a DNA exonuclease, 6) a DNA endonuclease, 7) an enzyme comprising RNaseH activity, and 8) one or more buffers suitable for one or more of the elements contained in the kit. In some embodiments, the kit further comprises a solid support (such as magnetic beads).

In some embodiments, the kit comprises an enzyme that cleaves RNA from an RNA/DNA hybrid, including but not limited to, RNase H or RNase I. In some embodiments, the kit further comprises a DNA polymerase, such as a DNA polymerase selected from the group consisting of a strand displacing DNA polymerase, a high-fidelity DNA polymerase, a polymerase that has proofreading activity, a T7 DNA polymerase, and an E. coli DNA polymerase. In some embodiments, the kit comprises a DNA ligase. In some embodiments, the kit comprises buffer suitable for any one of the reactions described herein, i.e., ligation, single-strand polynucleotide amplification, and enrichment, etc. These components may be provided in a separate kit, or provided together with the adaptors and primers described herein.

In some embodiments, the kit further comprises one or more probes, such as any of the probes described herein. In some embodiments, the kit comprise at least about 50, at least about 100, at least about 150, or more probes. The probes may be provided in a separate kit, or provided together with the adaptors and primers, or other reagents described herein.

The kits described herein may further comprise instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kits or components thereof (i.e., associated with the packaging or subpackaging) etc. In some embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

The various components of the kit may be in separate containers, where the containers may be contained within a single housing, e.g., a box.

Further provided herein are methods of making any of the articles of manufacture described herein.

### EXAMPLES

The following are examples of methods and compositions of the invention. It is understood that various other embodiments may be practiced, given the general description provided above.

### Example 1

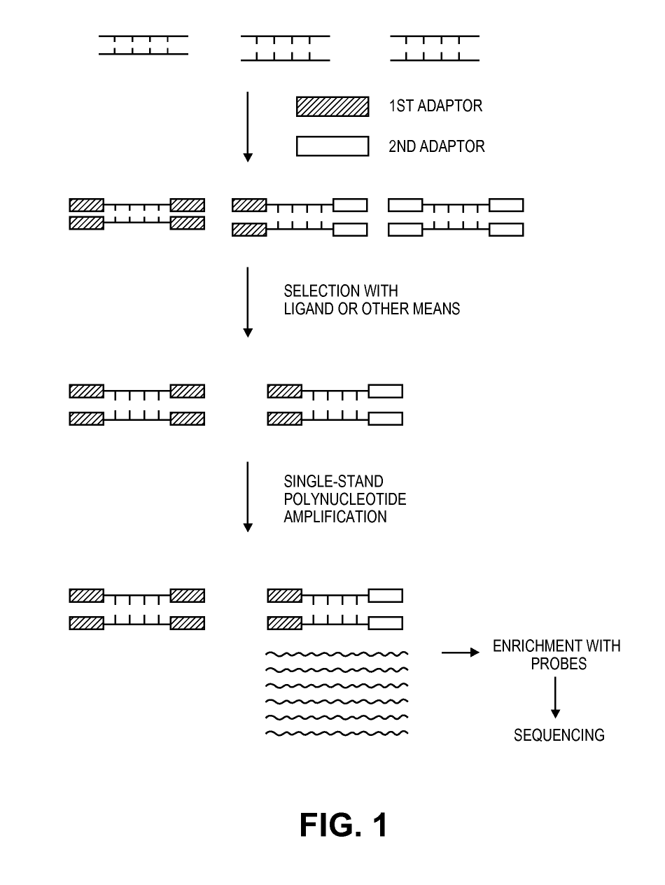

This example provides one exemplary method of processing genomic DNA for DNA sequencing using the asymmetric adaptor method. FIG. 1 provides a flow-chart for this method.

### Example 2

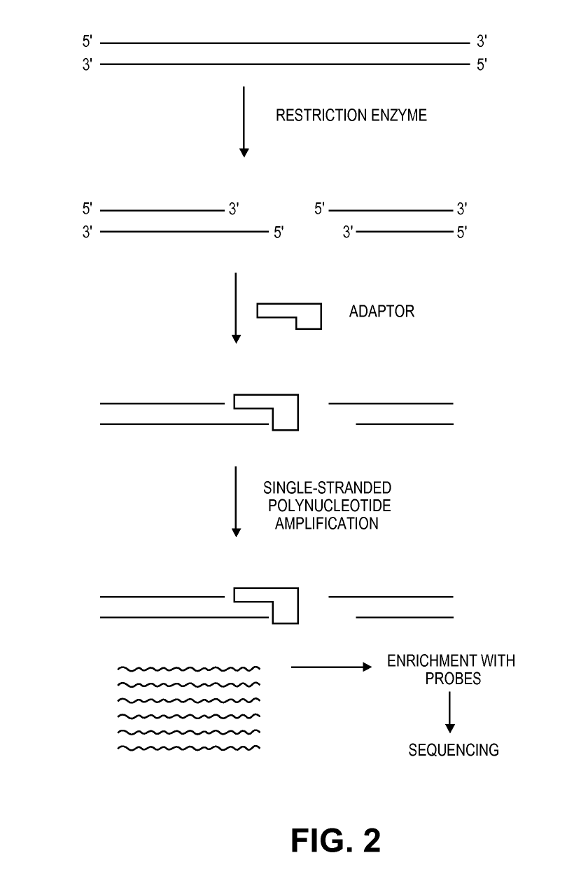

This example provides one exemplary method of processing genomic DNA for DNA sequencing using the restriction enzyme digestion method. FIG. 2 provides a flow-chart for this method.

1. A method of generating single-stranded polynucleotides comprising asymmetric adaptor sequences, comprising:
i) ligating one or more DNA fragments to: a) a first adaptor comprising a tag; and b) a second adaptor comprising a recognition sequence;
ii) selecting DNA fragments comprising the first adaptor based on the presence of the tag;
iii) amplifying the DNA fragments selected from step ii) by single-strand polynucleotide amplification using a primer comprising RNA and hybridizing the primer comprising RNA to the recognition sequence, thereby selectively amplifying DNA fragments comprising the second adaptor to obtain a population of single-stranded polynucleotides.

2. (canceled)

3. The method of claim 1, wherein one strand of the DNA fragment selected from step ii) is physically separated from its complementary strand before it is used as a template for the single-strand polynucleotide amplification.

4. A method of generating single-stranded polynucleotides comprising an adaptor sequence from a double-stranded target DNA, comprising:
i) cleaving the double-stranded target DNA with a restriction endonuclease to generate DNA fragments having a 5′ or 3′overhang;
ii) ligating the DNA fragments with an adaptor that comprises a) a single-stranded 5′ or 3′ overhang complementary to the 5′ or 3′ overhang of the DNA fragments and b) a recognition sequence; and
iii) amplifying the DNA fragments ligated to the adaptor by single-strand polynucleotide amplification using a primer comprising RNA and hybridizing the primer comprising RNA to the recognition sequence to obtain a population of single-stranded polynucleotides.

5-6. (canceled)

7. The method of claim 1, further comprising immobilizing the single-stranded polynucleotides on a solid support.

8. (canceled)

9. A method of analyzing one or more desired regions on a target polynucleotide, wherein the one or more desired regions are hybridizable to a set of probes, comprising:
1) contacting a population of single-stranded polynucleotides generated from said target polynucleotide with the set of probes;
2) separating polynucleotides that are bound to the probes from the rest of the polynucleotides, wherein polynucleotides comprising the one or more desired regions are enriched; and
3) analyzing the separated polynucleotides.

10. The method of claim 9, wherein the population of single-stranded polynucleotides is generated from said target polynucleotide by single-strand polynucleotide amplification using a primer comprising RNA and DNA fragments generated from said target polynucleotide as template.

11. The method of claim 9, wherein the one or more desired regions are regions where oncogenes are located.

12. The method of claim 9, wherein the set of probes comprises at least about 10 different polynucleotide probes.

13. (canceled)

14. The method of claim 9, wherein the target polynucleotide is RNA.

15. The method of claim 9, wherein the target polynucleotide is a double-stranded DNA.

16. The method of claim 10, wherein the population of single-stranded polynucleotides is generated by steps comprising:
i) ligating one or more DNA fragments generated from the target polynucleotide to: a) a first adaptor comprising a tag; and b) a second adaptor comprising a recognition sequence;
ii) selecting DNA fragments comprising the first adaptor based on the presence of the tag;
iii) amplifying the DNA fragments selected from step ii) by single-strand polynucleotide amplification using a primer comprising RNA and hybridizing the primer comprising RNA to the recognition sequence, thereby selectively amplifying DNA fragments comprising the second adaptor to obtain the population of single-stranded polynucleotides.

17. The method of claim 15, wherein the double-stranded DNA is genomic DNA.

18. The method of claim 9, wherein the analyzing comprises polynucleotide sequencing.

19. The method of claim 10, wherein the single-strand polynucleotide amplification comprises:
a) extending the primer comprising RNA in a complex comprising:
i) the DNA fragment to be amplified and
ii) the primer comprising RNA, wherein the primer comprising RNA is hybridized to the DNA fragment to be amplified; and
b) cleaving the RNA portion of the primer with an enzyme that cleaves RNA from an RNA/DNA hybrid such that another primer comprising RNA hybridizes to the DNA fragment and repeats primer extension by strand displacement;
whereby multiple copies of single-stranded polynucleotides are generated.

20. The method of claim 10, wherein the single-strand polynucleotide amplification comprises use of an RNA primer.

21. The method of claim 10, wherein the single-strand polynucleotide amplification comprises use of a DNA-RNA composite primer.

22. The method of claim 19, wherein the extension is carried out by a DNA polymerase selected from the group consisting of a strand displacing DNA polymerase, a high-fidelity DNA polymerase, a polymerase that has proofreading activity, a T7 DNA polymerase, and an E. coli DNA polymerase.

23. The method of claim 19, wherein the enzyme that cleaves RNA from the RNA/DNA hybrid is RNase H or RNase I.

24. A kit comprising i) a first adaptor comprising a tag; and b) a second adaptor comprising a recognition sequences, and iii) a primer that hybridizes to the recognition sequence.

25. The kit of claim 24, further comprising a ligand that binds to the tag.

26-37. (canceled)
